(19) United States
(12) Patent Application Publication
(10) Pub. No.: US 2011/0144502 A1
(43) Pub. Date: Jun. 16, 2011

(54) IMAGING GUIDEWIRE

(75) Inventors: Gan Zhou, City of Plano, TX (US); Jeffrey R. Gladden, City of Westlake, TX (US)

(73) Assignee: Tea Time Partners, L.P., Rockwall, TX (US)

(21) Appl. No.: 12/968,344

(22) Filed: Dec. 15, 2010

Related U.S. Application Data
(60) Provisional application No. 61/286,591, filed on Dec. 15, 2009.

(51) Int. Cl. A61B 8/12

(52) U.S. Cl. 600/463

(57) ABSTRACT

An imaging guidewire that, in one embodiment, includes: (1) a hypotube forming an elongated main body having a distal end, (2) at least one multimode optical fiber integral with the hypotube and configured to carry laser light for ultrasonic excitation, (3) a single-mode optical fiber integral with the hypotube, having a reflective coating located on a distal end thereof and at the distal end of the elongated main body and configured to carry laser light for ultrasonic detection and (4) an imaging cap coupled to the elongated main body at the distal end and including a photoacoustic layer configured to receive the laser light from the at least one multimode optical fiber.

### CROSS-REFERENCE TO RELATED APPLICATION

This application claims the benefit of U.S. Provisional Application Ser. No. 61/286,591, filed by Zhou, et al., on Dec. 15, 2009, entitled “Imaging Guidewire,” commonly assigned with this application and incorporated herein by reference.

### TECHNICAL FIELD

This application is directed, in general, to medical interventional procedures and more specifically to devices, taking the form of either catheters or guidewires.

### BACKGROUND

In interventional cardiology, catheters and guidewires are often inserted into a patient's artery or vein to help accomplish tasks such as angioplasty or pacemaker or defibrillator lead insertion. For example, a balloon dilation catheter expands at a site of blood vessel occlusion and compresses the plaque, improving patency of the vessel. An intravascular ultrasound (IVUS) catheter provides a 360° view of the lateral cross section of a vessel and is often used clinically to provide intravascular information on vessel condition and geometry. IVUS catheters can image through blood with an acceptable range and have become a successful diagnostic tool in interventional cardiology and other medical applications.

In IVUS, an ultrasonic transducer is embedded in a distal end of an imaging catheter. The catheter is advanced through a patient's vascular system to a target area. The transducer emits ultrasonic pulses and listens for echoes from the surrounding tissue. The echoes are used to form a one-dimensional (1D) image. The catheter can be rotated to obtain two-dimensional (2D) imaging data or, alternatively, a solid-state IVUS with an annular array of transducers at the catheter distal surface can be used to perform 2D image scanning. Combined with a controlled pullback motion, the device can also obtain three-dimensional (3D) image data in a cylindrical volume centered on the catheter.

While IVUS catheters are useful clinically, they must be used in conjunction with, and guided by, a guidewire. As such it is difficult for IVUS catheters to directly guide other therapeutic devices, such as stent deployment catheters. This limitation of IVUS catheter is mainly due to the size and stiffness of a typical IVUS catheter.

### SUMMARY

In one embodiment, the imaging guidewire includes: (1) a hypotube forming an elongated main body having a distal end, (2) at least one multimode optical fiber integral with the hypotube and configured to carry laser light for ultrasonic excitation, (3) a single-mode optical fiber integral with the hypotube, having a reflective coating located on a distal end thereof and at the distal end of the elongated main body and configured to carry laser light for ultrasonic detection and (4) an imaging cap coupled to the elongated main body at the distal end and including a photoacoustic layer configured to receive the laser light from the at least one multimode optical fiber.

In another embodiment, the imaging guidewire includes: (1) a hypotube forming an elongated main body having a distal end, (2) at least one multimode optical fiber integral with the hypotube and configured to carry laser light for ultrasonic excitation, (3) a single-mode optical fiber integral with the hypotube, having a reflective coating located on a distal end thereof configured to carry laser light for ultrasonic detection, (4) an imaging cap located in the elongated main body other than at the distal end and including a photoacoustic layer configured to receive the laser light from the at least one multimode optical fiber and (5) a flexible guidewire coupled to the hypotube and extending from the distal cap.

In another embodiment, the imaging guidewire includes: (1) a hypotube forming an elongated main body having a distal end, (2) a plurality of multimode fibers integral with the hypotube and configured to carry laser light for ultrasonic excitation, (3) a single-mode optical fiber integral with the hypotube, having a reflective coating located on a distal end thereof and at the distal end of the elongated main body and configured to carry laser light for ultrasonic detection, the plurality of multimode fibers generally centered around the single-mode optical fiber and (4) an imaging cap coupled to the elongated main body at the distal end including an inner through-hole having a diameter based on a diameter of the single-mode optical fiber and a photoacoustic layer configured to receive the laser light from the at least one multimode optical fiber, the glass collar further having a generally flat end-face surface and a generally conical opposing end-face surface.

### BRIEF DESCRIPTION

Reference is now made to the following descriptions taken in conjunction with the accompanying drawings, in which:

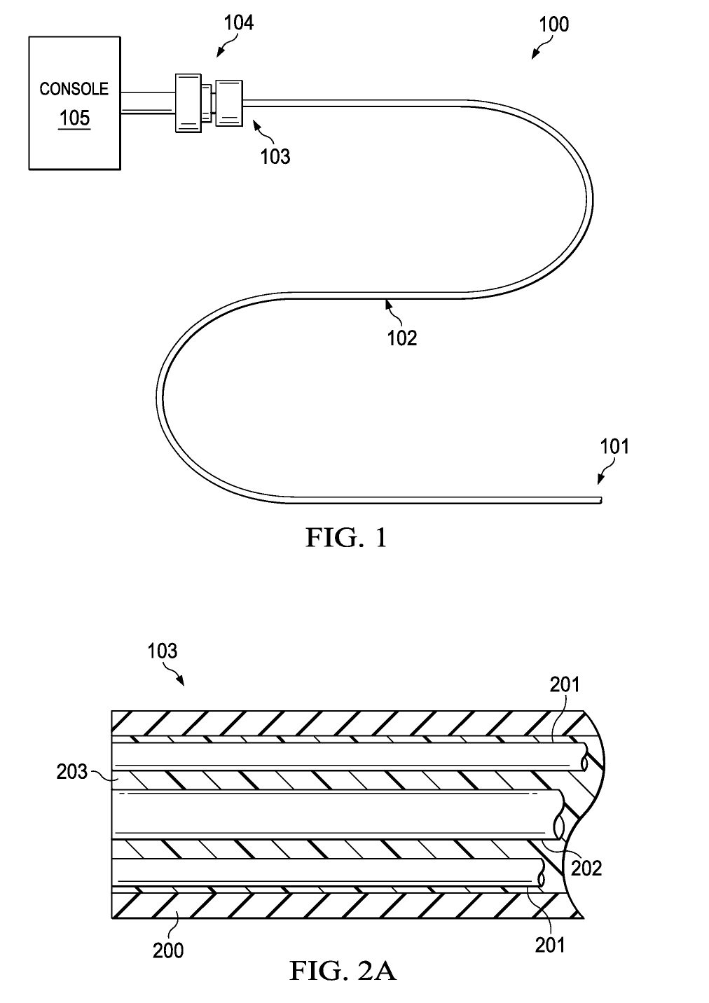

FIG. 1 is a schematic view of one embodiment of an imaging guidewire constructed according to the principles of the invention;

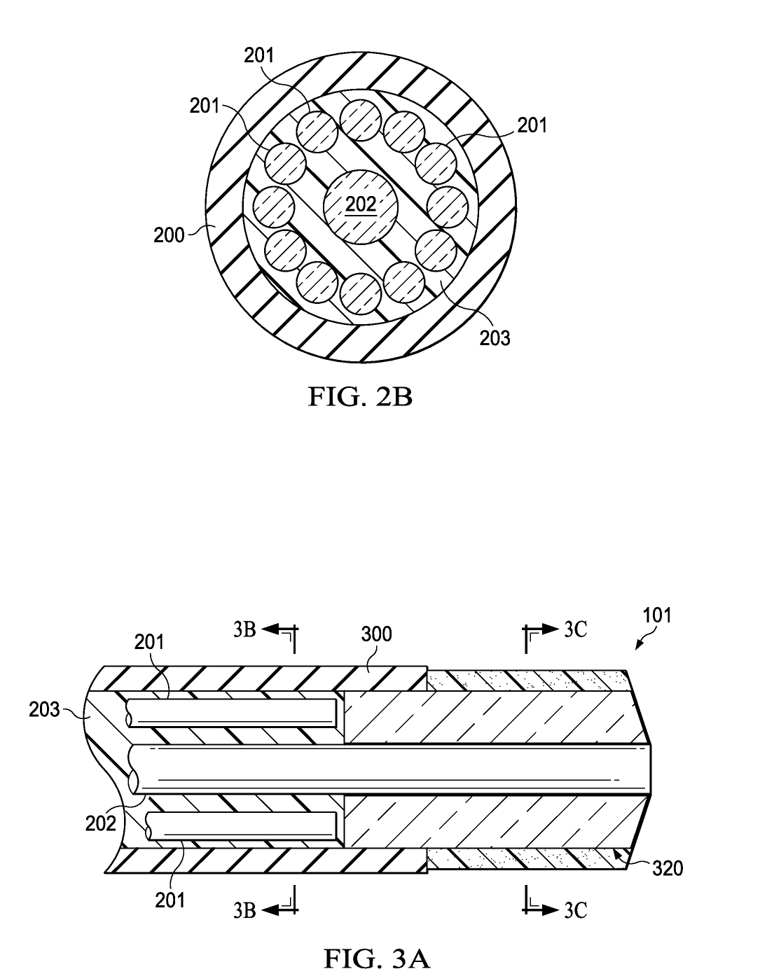

FIG. 2 is a schematic view of one embodiment of a proximal end of the imaging guidewire of FIG. 1;

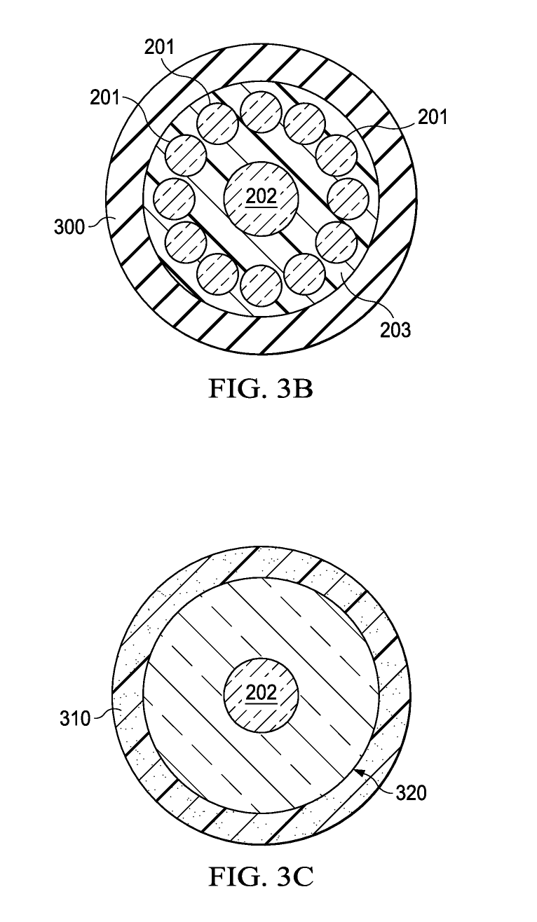

FIGS. 3A-C are schematic views of one embodiment of a distal end of the imaging guidewire of FIG. 1;

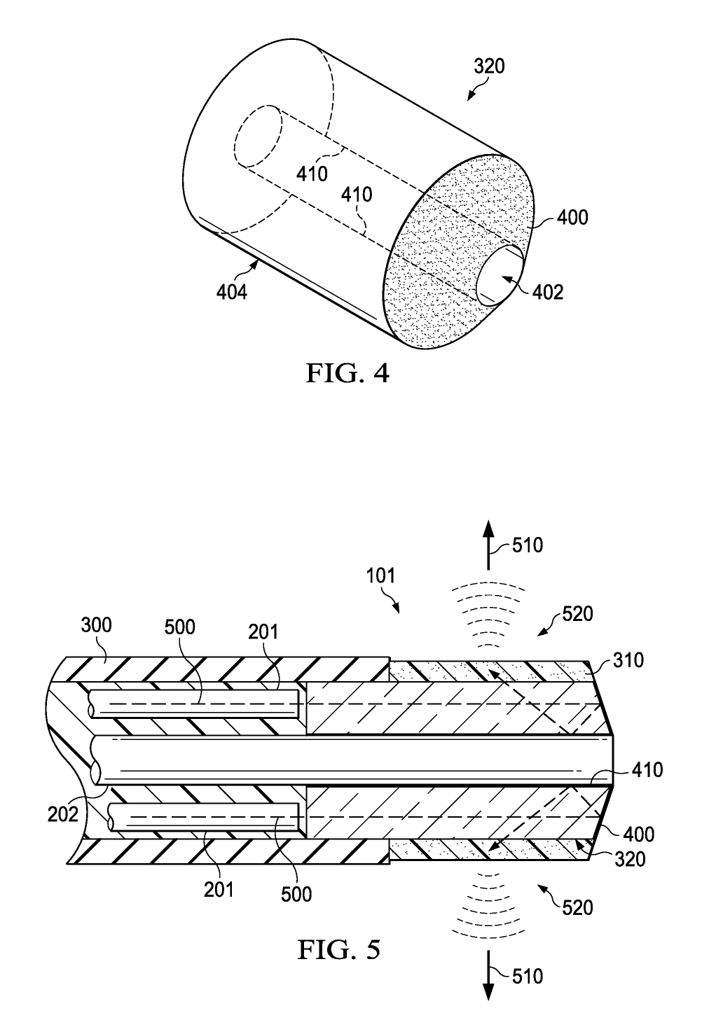

FIG. 4 is a schematic view of one embodiment of an imaging cap of the imaging guidewire of FIG. 1;

FIG. 5 is a schematic view of one example of light propagation and ultrasound excitation that may occur within the imaging cap of FIG. 4;

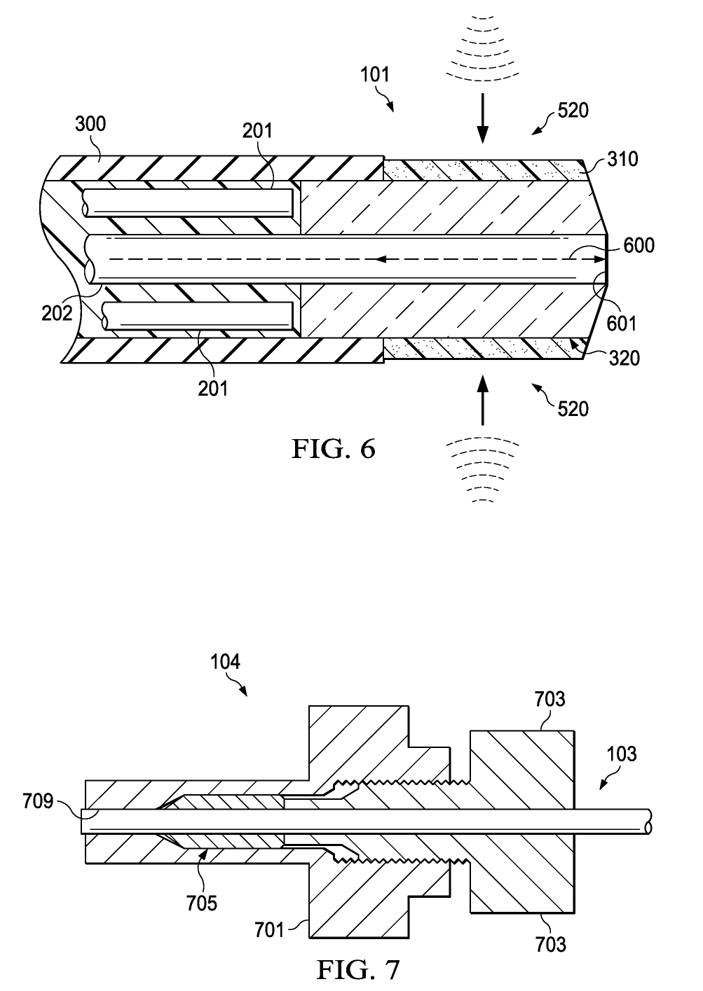

FIG. 6 is a schematic view of one example of light propagation that may occur within, and ultrasound detection that may occur by, a single-mode optical fiber in the imaging guidewire of FIG. 1;

FIG. 7 is a cross-sectional view of one embodiment of a guidewire connector;

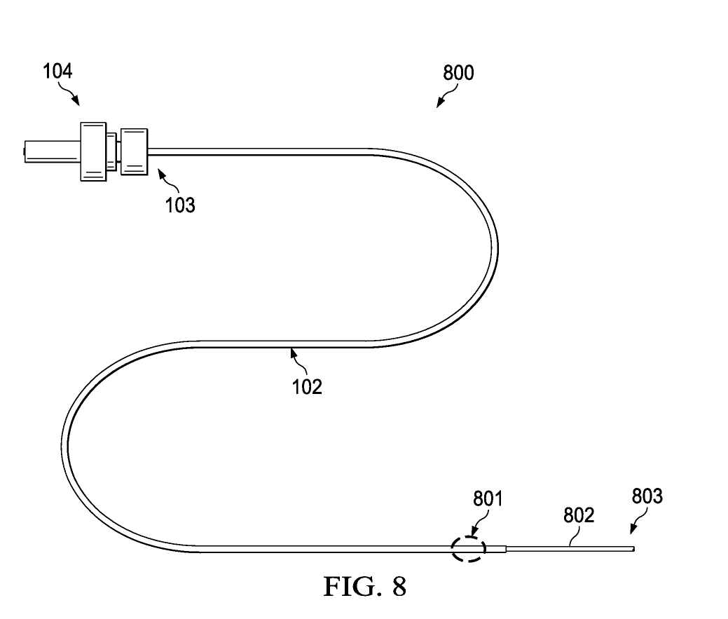

FIG. 8 is a schematic view of another embodiment of an imaging guidewire in which an imaging element thereof is located before the distal end of the guidewire; and

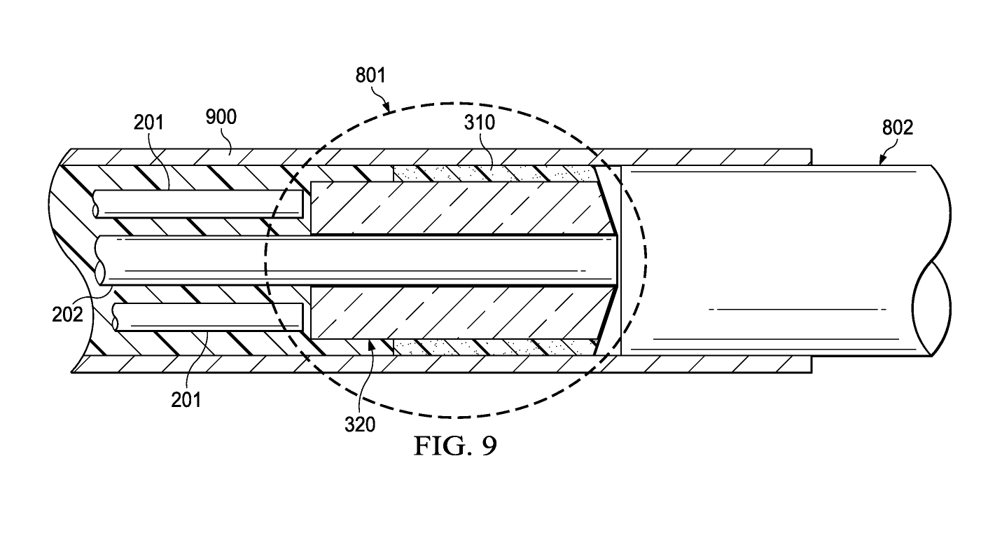

FIG. 9 is a schematic view of one embodiment of a body section of the imaging guidewire of FIG. 8.

### DETAILED DESCRIPTION

Imaging guidance of therapeutic catheters would be helpful for difficult cases such as ostium stenting, where it is desirable to deploy the stent at a precise location in the vessel. What is needed is an imaging guidewire that can perform IVUS-like imaging function, yet can still work as a conventional wire that can be used to guide other catheters.

International application WO2006/030408 titled “Intravascular Ultrasound Imaging Device,” by Matcovitch, et al., describes an optical fiber adapted for ultrasound imaging. However, Matcovitch does not adequately address the two important aspects of an imaging guidewire as pointed out above, that is, the imaging aspect and the guidewire aspect. To perform imaging, Matcovitch requires the use of a group of optical wavelength filters built into an adapted optical fiber, together with a pulsed light source with tunable wavelength, or equivalently, a group of pulsed light sources with different wavelengths. As a result, the device is complex and difficult to manufacture. Secondly, the complex optical fiber design of a ring core integrated with an inner core makes the total optical fiber diameter large. As is well-known in art, the stiffness of a glass optical fiber goes up roughly as the fourth power of its diameter. So the specific construction of Matcovitch's optical fiber makes it mechanically stiff, rendering it difficult to use as a guidewire, since flexibility and the ability to track complex vessel geometry is a must for a guidewire.

Various embodiments of a novel imaging guidewire will be described herein that address one or more of the deficiencies or needs set forth above. Various structures, arrangements, relationships and functions may be asserted as being associated with or necessary to certain of the several embodiments. Those skilled in the pertinent art should understand, however, that those structures, arrangements, relationships and functions need not be associated with or necessary to the invention in its broad form.

Referring initially to FIG. 1, illustrated is a schematic view illustrating one embodiment of an imaging guidewire 100 constructed according to the principles of the invention. The imaging guidewire 100 includes an elongated main body 102, a connector 104 located at a proximal end 103 of the main body 102 and an imaging cap 101 coupled to the main body 102 at an unreferenced distal end of the main body 102. The connector 104 is configured to be coupled to a console 105. The illustrated embodiment of the console 105 is configured to generate, receive and analyze (e.g., determine phase shifts in) laser light. Other embodiments of the console 105 perform other functions. A plurality of multimode optical fibers (to be shown and referenced in a more specific embodiment in FIG. 2) is integral with (e.g., embedded in) the main body 102.

FIGS. 2A and 2B together show one embodiment of an arrangement of a plurality of multimode optical fibers 201 inside the main body 102 of the imaging guidewire 100 at its proximal end 103. FIG. 2A shows the cross-sectional view of the proximal end 103 (without showing the connector 104), in a plane containing the longitudinal axis of the imaging guidewire 100. FIG. 2B shows the cross-sectional view looking into the main body 102 of the imaging guidewire 100 in a plane perpendicular to the longitudinal axis of the imaging guidewire 100. The proximal end 103 comprises a hypotube 200, the plurality of multimode optical fibers 201, at least one single-mode optical fiber 202, and a fillant (e.g., epoxy) 203. In the illustrated embodiment, the plurality of multimode optical fibers 201 are approximately evenly spaced inside the hypotube 200 and generally centered around the single-mode optical fiber 202.

At the proximal end 103 of the wire, two types of lasers (not shown) are configured to direct laser energy into the optical fibers 201, 202. More specifically, in the illustrated embodiment, a first laser (e.g., a pulsed laser) is configured to provide laser energy into the plurality of multimode optical fibers 201. Likewise, in the illustrated embodiment, a second laser (e.g., a continuous-wave laser) is configured to provide laser energy into the single-mode fiber 202. With the help of a scanner (such as a galvanometer, not shown) the pulsed laser energy can be coupled to all the multimode optical fibers in a sequential way.

FIGS. 3A-C show one embodiment of the distal end of the imaging guidewire 100 and the imaging cap 101 thereof, with a few cross-sectional views. FIG. 3A shows the cross-sectional view of the distal end in a plane containing the longitudinal axis of the wire. The plurality of multimode optical fibers 201 are seen to terminate proximate, but short of, the distal end proximate the imaging cap 101. In contrast, the single-mode optical fiber 202 extends beyond the plurality of multimode optical fibers 201 and terminates at the distal end 101 of the imaging guidewire 100. A cross-sectional view A-A illustrated in FIG. 3B shows that the multimode optical fibers are approximately evenly spaced inside a hypotube 300 and centered around the single-mode optical fiber 202. A cross-sectional view B-B illustrated in FIG. 3C shows one example embodiment of the imaging cap 101, comprising a glass collar 320 and a photoacoustic layer 310. The photoacoustic layer 310 can be, for example, a suitable polymer material mixed with appropriate light-absorbing particles. The photoacoustic layer 310 is configured to absorb laser pulse energy incident on it and generate an ultrasonic wave accordingly.

FIG. 4 is an isometric view of an example embodiment of the glass collar 320 that is part of the imaging cap 101. In this embodiment, the glass collar 320 takes the form of a generally cylindrically-shaped glass element, with an inner through-hole 402, which in the illustrated embodiment is of the same geometry as the single-mode optical fiber 202, i.e., cylindrical. Likewise, in the illustrated embodiment, the diameter of the inner through-hole 402 is based on (e.g., closely matches) that of the single-mode optical fiber 202 while allowing the single-mode optical fiber to be inserted into it. The illustrated embodiment of the glass collar 320 has a generally flat surface as one of its end faces and a generally conical surface as the other of its end faces. Other embodiments have other opposing end-face configurations (e.g., concave conical and rounded). The illustrated embodiment of the conical end face has a reflective coating 400 located on it. The side wall of the through-hole 402 can also have a reflective layer 410 deposited on it, such as a layer of silver, or a layer of low refractive index material that enables total internal reflection between the glass collar 320 and the layer 410. In the illustrated embodiment, the outer surface 404 of the glass collar 320 has no other coating, except for the photoacoustic layer 310 (not shown in FIG. 4) that is deposited on it. Other embodiments have additional coatings, e.g., to protect the photoacoustic layer 310 from oxidation or damage from mechanical contact with other objects.

FIG. 5 illustrates an embodiment of the principle of ultrasound generation using the imaging cap 101 constructed according to the principles of the invention. A laser pulse 500 carried by a multimode optical fiber 201 exits the multimode optical fiber 201 proximate the imaging cap 101, and subsequently enters the glass collar (unreferenced in FIG. 5) of the imaging cap 101. The laser pulse 500, upon reaching the conical end of the glass collar, is reflected by the reflective coating 400. The reflected light is then further reflected by the reflective coating 410 on the inner wall of the glass collar, to reach the photoacoustic layer 310. The photoacoustic layer 310 absorbs the laser pulse 500, causing an ultrasonic wave 510 to be generated and propagated into surrounding media (typically blood or tissue for an intravascular application) surrounding the imaging cap 101. An ultrasonic echo 520 from an object in the media can then propagate back toward the imaging cap 101 and be detected by the single-mode optical fiber 202, as explained below. Different sections of the photoacoustic layer 310 can be excited to generate ultrasonic wave by coupling the laser pulse 500 to different multimode optical fibers, thereby making it possible to perform pulse-echo detection for a 360° range of angles surrounding the imaging cap 101.

FIG. 6 illustrates an embodiment where the ultrasonic echo 520 can be detected by the single-mode optical fiber 202. A continuous-wave laser 600 is optically coupled to the single-mode optical fiber 202 at the proximal end 103 of the imaging guidewire 100. Laser light 600 propagates in the single-mode optical fiber 202 toward the distal end of the imaging guidewire, where it is reflected by a coating 601 deposited at the distal end of the single-mode optical fiber 202. The reflected light 600 back-propagates in the single-mode optical fiber 202 toward the proximal end 103 of the imaging guidewire 100, where it exits the imaging guidewire 100 and enters a console (not shown in FIG. 6). The ultrasonic echo 520 can cause phase modulation on the reflected light 600, and this modulation can be detected in a number of ways using an optical interferometer (not shown). A detailed explanation of various embodiments of such an ultrasonic sensor can be found in U.S. Patent Application 2010/0199773 A1, filed on Apr. 16, 2010, by Zhou, entitled “Method and Apparatus for Noise Reduction in Ultrasound Detection” (incorporated herein by reference) and the references cited therein.

FIG. 7 illustrates the cross-section view of an embodiment of a connector 104 where the proximal end 103 of the imaging guidewire terminates. The connector 104 can have a ferrule 701 with a bore 709 through which the proximal end 103 can be inserted. A compression nut 703 is configured to thread onto the ferrule 701 and press against a compression sleeve 705. The compression sleeve 705 will then engage the proximal hypotube (e.g., the hypotube 200 of FIG. 2) of the imaging guidewire and grip and hold the tube securely in the ferrule 701. In one embodiment the ferrule 701 can be magnetized so it can attach to a mating receptacle (not shown) on a console (not shown in FIG. 7). Such a connector 104 allows the imaging wire 100 for insertion into and detachment from a console (e.g., the console 105 of FIG. 1). It also gives a user the option to pull the proximal end 103 out of the connector (by loosening the compression nut 703) in case the imaging guidewire 100 is to be used as a conventional guide for other catheters and the clinician needs access to the proximal tip of the wire.

FIG. 8 illustrates another embodiment of an imaging guidewire 800 constructed according to the principles of the invention. The imaging guidewire 800 shares many of the features of the imaging guidewire 100 of FIG. 1. The imaging guidewire 800 has a proximal end 103, a connector 104 coupled to the proximal end 103, a main body 102 and an imaging element 801. The principal difference between the imaging guidewire 800 and the imaging guidewire 100 is that the imaging guidewire 800 has an imaging element 801 at a location other than a distal end 803 of the main body 102, whereas the imaging cap 101 of the imaging guidewire 100 is located at its distal end. In addition, the distal section 802 of the imaging guidewire 800 has a flexible body similar to a conventional guidewire, and can optionally have a floppy tip 803 if so desired. An imaging wire such as the imaging guidewire 800 could be useful in some difficult cases such as ostium stenting, where the imaging function of the wire can be used to precisely pinpoint a target location in the vessel, and the distal section helps to anchor the wire to assist the positioning and deployment of a stent.

FIG. 9 shows an example embodiment of the imaging element 801 of the imaging guidewire 800. The illustrated embodiment has generally the same construction as the imaging cap of the imaging guidewire 100 of FIG. 1, except that the hypotube 900 extends beyond the imaging element 801 and is attached to a flexible conventional guidewire 802, as illustrated. The hypotube 900 can be made of a suitable material that can transmit the ultrasound signal of interest. Such material can be an appropriate type of polymer, for example. In one embodiment, the hypotube 900 is composed of metal with windows micro-machined on its surface to allow ultrasound to transmit through efficiently.

Those skilled in the art to which this application relates will appreciate that other and further additions, deletions, substitutions and modifications may be made to the described embodiments.

What is claimed is:

1. An imaging guidewire, comprising:
a hypotube forming an elongated main body having a distal end;
at least one multimode optical fiber integral with said hypotube and configured to carry laser light for ultrasonic excitation;
a single-mode optical fiber integral with said hypotube, having a reflective coating located on a distal end thereof and at said distal end of said elongated main body and configured to carry laser light for ultrasonic detection; and
an imaging cap coupled to said elongated main body at said distal end and including a photoacoustic layer configured to receive said laser light from said at least one multimode optical fiber.

2. The guidewire as recited in claim 1 wherein said at least one multimode optical fiber is a plurality of multimode fibers generally centered around said single-mode optical fiber.

3. The guidewire as recited in claim 1 wherein said imaging cap comprises a glass collar including an inner through-hole having a diameter based on a diameter of said single-mode optical fiber, said glass collar further having a generally flat end-face surface and a generally conical opposing end-face surface.

4. The guidewire as recited in claim 1 further comprising a fillant embedding said at least one multimode optical fiber and said single-mode optical fiber within said hypotube.

5. The guidewire as recited in claim 1 further comprising a connector coupled to a proximal end of said elongated main body.

6. The guidewire as recited in claim 1 wherein said photoacoustic layer comprises a polymer material mixed with light-absorbing particles.

7. The guidewire as recited in claim 1 wherein said reflective coating is composed of silver.

8. An imaging guidewire, comprising:
a hypotube forming an elongated main body having a distal end;
at least one multimode optical fiber integral with said hypotube and configured to carry laser light for ultrasonic excitation;
a single-mode optical fiber integral with said hypotube, having a reflective coating located on a distal end thereof configured to carry laser light for ultrasonic detection;
an imaging cap located in said elongated main body other than at said distal end and including a photoacoustic layer configured to receive said laser light from said at least one multimode optical fiber; and
a flexible guidewire coupled to said hypotube and extending from said distal cap.

9. The guidewire as recited in claim 8 wherein said at least one multimode optical fiber is a plurality of multimode fibers generally centered around said single-mode optical fiber.

10. The guidewire as recited in claim 8 wherein said imaging cap comprises a glass collar including an inner through-hole having a diameter based on a diameter of said single-mode optical fiber, said glass collar further having a generally flat end-face surface and a generally conical opposing end-face surface.

11. The guidewire as recited in claim 8 further comprising a fillant embedding said at least one multimode optical fiber and said single-mode optical fiber within said hypotube.

12. The guidewire as recited in claim 8 further comprising a connector coupled to a proximal end of said elongated main body.

13. The guidewire as recited in claim 8 wherein said photoacoustic layer comprises a polymer material mixed with light-absorbing particles.

14. The guidewire as recited in claim 8 wherein said reflective coating is composed of silver.

15. An imaging guidewire, comprising:
a hypotube forming an elongated main body having a distal end;
a plurality of multimode fibers integral with said hypotube and configured to carry laser light for ultrasonic excitation;
a single-mode optical fiber integral with said hypotube, having a reflective coating located on a distal end thereof and at said distal end of said elongated main body and configured to carry laser light for ultrasonic detection, said plurality of multimode fibers generally centered around said single-mode optical fiber; and
an imaging cap coupled to said elongated main body at said distal end including an inner through-hole having a diameter based on a diameter of said single-mode optical fiber and a photoacoustic layer configured to receive said laser light from said at least one multimode optical fiber, said glass collar further having a generally flat end-face surface and a generally conical opposing end-face surface.

16. The guidewire as recited in claim 15 further comprising a fillant embedding said plurality of multimode fibers and said single-mode optical fiber within said hypotube.

17. The guidewire as recited in claim 15 further comprising a connector coupled to a proximal end of said elongated main body.

18. The guidewire as recited in claim 15 wherein said photoacoustic layer comprises a polymer material mixed with light-absorbing particles.

19. The guidewire as recited in claim 15 wherein said reflective coating is composed of silver.
